(19) United States
(12) Patent Application Publication
(10) Pub. No.: US 2007/0299298 A1
(43) Pub. Date: Dec. 27, 2007

(54) Multisensory animated picture

(75) Inventors: David Suissa, Vincennes (FR); Clement Jeanjean, Paris (FR)

Correspondence Address: HARNESS, DICKEY & PIERCE, P.L.C., P.O. BOX 828, BLOOMFIELD HILLS, MI, 48303, US

(73) Assignee: Presensia, Paris (FR)

(21) Appl. No.: 11/788,041

(22) Filed: Apr. 18, 2007

Related U.S. Application Data
(63) Continuation of application No. PCT/FR05/02600, filed on Oct. 19, 2005.

(30) Foreign Application Priority Data
Oct. 19, 2004 (FR) 04/11097

(51) Int. Cl. A61M 21/02

(52) U.S. Cl. 600/27

(57) ABSTRACT

The invention enables digital works combining fixed images, animated images, sounds and smells to be disseminated to an audience. The multisensory picture looks like a traditional art picture with a removable frame which is personalized in harmony with the work thus disseminated. A high-resolution flat electronic screen (1) occupies the space instead of the picture. Integrated devices enable sounds (2) and smells (3) chosen by the creators of the multisensory works to be disseminated. The invention activates human senses such as sight, hearing, smell and possibly touch, enabling viewers to experience strong emotions.

The present invention relates to a multi-sensorial broadcasting device comprising multimedia broadcasting means (images and sounds) and scent broadcasting means. Such a picture is known from patent application WO 92/13474 in which an artificial window comprises different means of lighting, projection and production of sounds and scents so as to produce sensory effects for a spectator. However, this device with advantageous sensory effects has certain drawbacks, in particular the fact that it does not allow for the broadcasting of elaborate multimedia digital works which have olfactory effects associated with them.

A scent-storage device and a scent-broadcasting system based on this device are also known from patent U.S. Pat. No. 6,602,475. The particularity of this device and of this system lies in the fact that the scent media and the multimedia data are contained in the same medium and are therefore closely linked. One drawback of this solution is the cost of producing such media as well as the exhaustible nature of the scent media which renders the medium, and therefore the multimedia content, perishable.

A device for broadcasting scents to a user in conjunction with video images and/or sounds is also known from patent U.S. Pat. No. 6,169,595. A flow of air passes through one or more valves to create a mixture of scents, which is transmitted to the nose of the user by means of a tube. This device has the drawback of being dedicated to a single user.

A display system which is intelligent in relation to the presence of people around it is also known from patent application US 2001/038034. Such a device does not relate to multi-sensory broadcasting, in particular cumulative broadcasting of scents, sound or digital graphic media.

The invention aims to solve at least one of these drawbacks. For this purpose, the invention relates to a device for broadcasting digital media combining still images, animated images, sounds, scents and possibly touch to the public Henceforth referred to as “multi-sensory picture”, the invention is presented in the form of a standard picture, the frame of which can be removed and personalised according to the digital work being broadcast. The picture canvas is replaced by a high-resolution electronic flat screen and an integrated device enables the broadcasting of different scents chosen in accordance with the sequence of images displayed and sounds emitted. The invention thus stimulates the human senses, namely sight, hearing, smell and even touch. In relation to the preceding, the frame can advantageously have raised patters which are pleasing to touch and designed in harmony with the all the images, sounds and scents broadcast. When used in the field of catering, the multi-sensory pictures can also be associated with dispensers of edible products so as to also stimulate the fifth human sense, namely taste.

The multi-sensory picture makes it possible to broadcast specific digital works created by artists and designed to provoke emotions by simultaneous stimulation of the senses. For this purpose, works intended to be broadcast by means of the invention will be designed so that the images, sounds, scents and frame form an inseparable whole. In particular, the images, sounds and scents preferably change in a synchronised manner.

The interchangeable frame is made from moulded wood, for example, similar to a standard picture frame, with drawings or raised patters that are specific to the work being broadcast. Thus, the multi-sensory picture is more of a picture with extended functionality than a standard multimedia object such as a television, a computer or a multimedia telephone, even when these objects are associated with a scent diffuser as in the case of patent WO0018696A1.

The multi-sensory picture is different from the invention set forth in patent EP01212104B1, which consists of a scent broadcasting accessory device controlled by a digital television or a computer. It is also different from the invention described in patent WO00186986A1 which relates to the field of mobile telephony. Furthermore, scent diffusing devices exist which are designed to odorise large rooms, for example railway station concourses, in which screens for displaying information or even animated images can also be placed. These devices generally diffuse a single scent within large spaces, and no synchronicity or harmony is sought between the broadcasting of scent and the broadcasting of data. Also available on the market are scent diffusers designed for individual work stations. In this case a single scent is generally diffused in a very localised manner. These devices are used, for example, for broadcasting over a very short distance a smell on request coming from internet-accessible sites, known as olfactory sites.

In the case of the multi-sensory picture, the personalised frame and the set of various scents are specifically chosen to match the audiovisual content of the work, which causes all the still images, animated images, sounds, scents and the frame to form an inseparable whole, specific to each multi-sensory work created, which can only be broadcast by means of the invention. The multi-sensory picture enables the broadcasting of digital works which cannot be broadcast by means of devices known in the state of the art.

The present invention, in its broadest sense, relates to a multi-sensory picture comprising graphic display means, sound broadcasting means, means of broadcasting a number of scents, digital data-reception means and computer means, said computer means being arranged to control and synchronise said display, sound broadcasting and scent broadcasting means.

In one embodiment of the invention, the frame can be removed and exchanged, allowing it to be personalised according to the digital multi-sensory work broadcast.

In another embodiment, the scent broadcasting means work by a method of micronising an odorous liquid, liquids with different scents being placed in different refillable or replaceable vials.

In one embodiment, the scent broadcasting means work by causing an airflow to pass through a series of porous balls previously soaked in odorous liquids, balls with different scents being placed in different refillable or replaceable containers. These two broadcasting means combine easy management of scent modules, by simply replacing the vials or balls, with precise and effective broadcasting of scents by micronisation or controlling the airflow passing through them.

In one specific embodiment, the sound source of the sound broadcasting means is mostly concealed and emits the sound in the direction of a rigid plate forming a reflector, allowing the sound to propagate towards the spectator without having to pass through the picture. In one embodiment, the multi-sensory picture comprises means of downloading the digital multi-sensory work to be broadcast, by using a wired or wireless link of known type, thereby providing access to a large number of digital works.

In one embodiment, the computer means comprise a program for counting the number of times the multi-sensory works are broadcast, with a view to calculating the royalties owed to their creators.

In an alternative embodiment, the multi-sensory picture comprises mechanical means that automatically orient the picture in portrait, landscape or diagonal format, controlled by the computer means according to commands contained in the digital multi-sensory work. This system makes it possible automatically to adapt to the format of the digital work and therefore to guarantee correct visual presentation.

In one specific embodiment, the computer means comprise software for decrypting previously encrypted digital multi-sensory works, so as to prevent a multi-sensory work from being broadcast to users not owning the software decryption key.

In another embodiment, a plurality of multi-sensory pictures communicate over a standard telecommunications network with a remote computer server, making it possible to broadcast digital multi-sensory works in several locations from a single server.

In an alternative embodiment, a plurality of multi-sensory pictures communicate with each other by means of a wired or wireless link, making it possible to broadcast multi-picture multi-sensory works in the same room.

In one specific embodiment, the animated multi-sensory pictures comprise a software database intended to design multi-sensory works.

According to various embodiments:
the multi-sensory picture comprises means for carrying a flow of air loaded with scent molecules through said scent broadcasting means, said duct means comprising an outlet orifice located below said screen and pointing substantially upwards,
said outlet orifice is at an angle greater that zero in relation to the horizontal, upwards in the direction of the spectator,
said screen is mounted on a door mounted such as to pivot in relation to the frame, which comprises all the computer, scent broadcasting, sound broadcasting and digital data-reception means,
the multi-sensory picture comprises means capable of placing said scent broadcasting means in neutral position and locking said containers of said scent broadcasting means when the door is open in relation to said frame.

The invention will also be better understood in reference to the following figures, wherein the same references are used to refer to identical or similar elements:

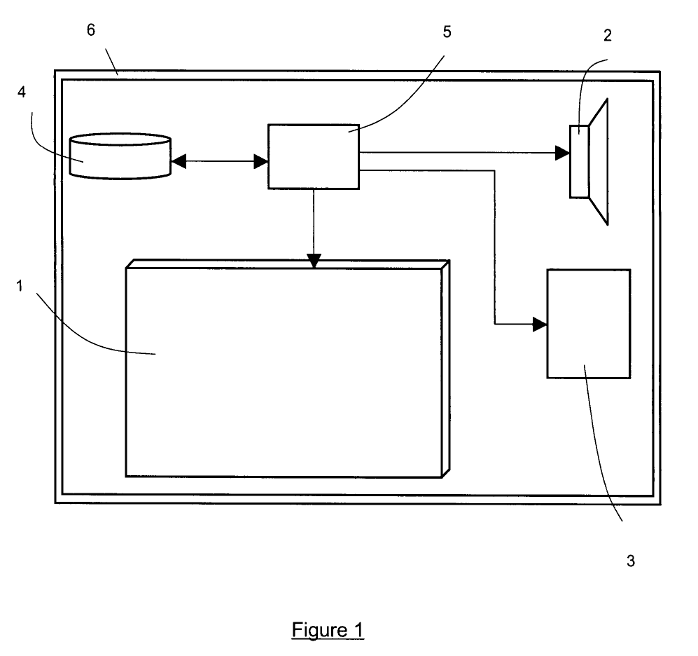

FIG. 1 shows an overview diagram of the main components of the invention, namely a screen for displaying still or animated images (1), sound broadcasting means (2), means of broadcasting a plurality of scents (3), data-reception means (4), computer means (5) and an interchangeable frame (6).

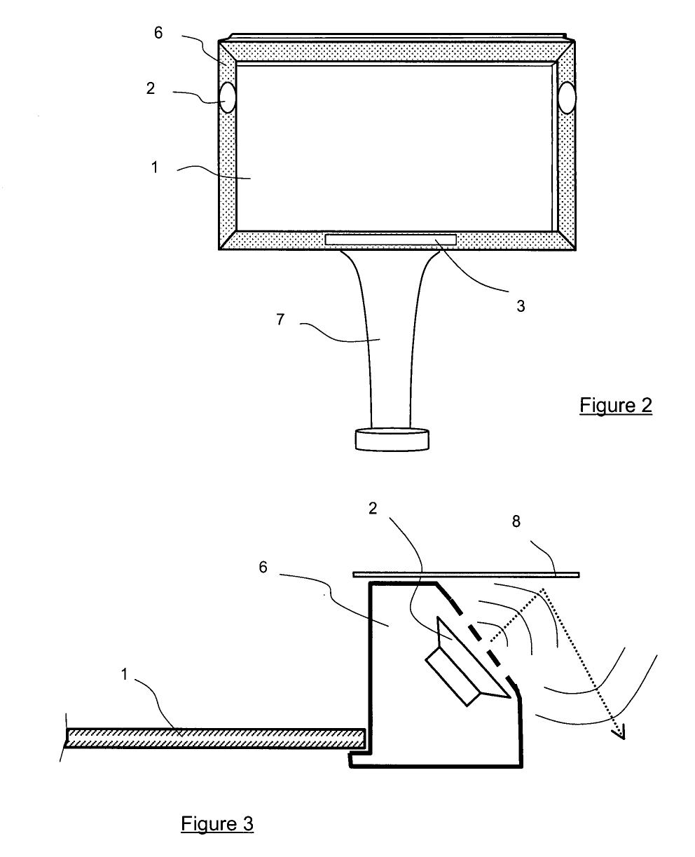

FIG. 2 shows an example of a multi-sensory picture design in so-called “landscape” format, mounted on a stand (7).

FIG. 3 shows an example of a design of sound broadcasting means (2) which stop the sound from having to pass through the picture although the sound source is visually concealed by the latter.

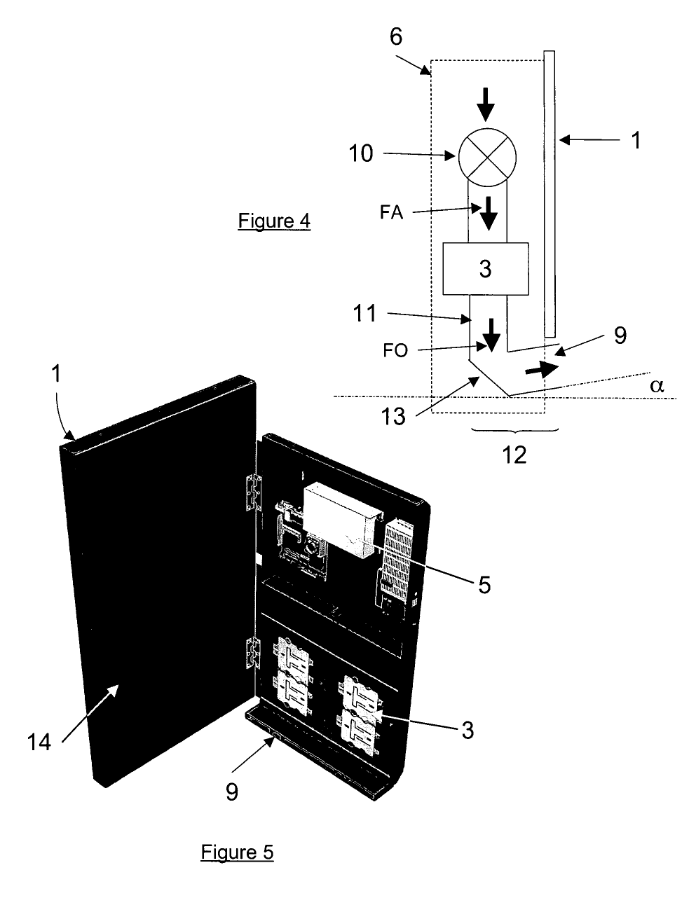

FIG. 4 shows the accessibility of the computer and scent broadcasting elements inside the picture.

The picture preferably has a diagonal dimension of between 60 and 110 cm and a thickness less than 10 cm. The flat electronic display screen (1) uses LCD technology, with a large screen and high resolution, for example 70 cm along the diagonal and 2 million pixels. For larger sizes, the screen advantageously uses plasma technology.

The sound broadcasting means (2) can consist of one or more standard membrane loudspeakers, implement piezoelectric technology, or use any other technology already known in the state of the art, when it can be integrated within the dimensions of the frame (6), or, more generally, when it can be integrated in a scarcely visible manner. It is known that the diameter of the sound source must be relatively large if high-quality sound is to be obtained, even for low frequencies of the audible spectrum, for example frequencies between 50 and 200 Hertz. As it is desirable for the sound source be scarcely visible due to aesthetic constraints, one design involves placing this source in such a way that it is concealed by the multi-sensory picture. However, in this case, the sound is degraded when passing through the material of the picture. A solution to compensate for this drawback involves placing the sound source in a visibly concealed manner behind the picture, for example in the frame, and pointing it towards a rigid plate (8) forming a reflector for the sound, so that the sound thus reflected is propagated towards the user without having to pass through the picture.

The scent broadcasting means (3) preferably comprise several containers, for example four containers, each containing small porous balls soaked in an odorous liquid. The scents in each container are different from the rest and their composition is chosen during the creation of the multi-sensory work in order to match the broadcast images and sounds. An electric turbine causes the air to circulate through one or more of the containers and to diffuse this air, thus loaded, towards the front of the multi-sensory picture. When the balls dry up, it is sufficient to replace the corresponding container, reload it with new soaked balls or re-soak the dry balls. Only one scent is broadcast at any given time, which can be obtained by causing the air to circulate through only one of the containers, the others being closed, for example by means of solenoid valves controlled by the computer unit (5).

Various other known techniques can be implemented to design the scent broadcasting means (3), for example that described in patent EP01054697B1. Another technique involves micronising odorous liquids placed in refillable or replaceable vials. This involves forcing the liquid through a hole with a small diameter under high pressure created by a compressor. Microdroplets are then formed, which quickly vaporise in contact with the air, releasing the scent molecules. Another known micronisation method involves using the mechanical vibrational force of piezoelectric material subjected to an alternating current in order to create the microdroplets. Any other micronisation method known in the state of the art can be implemented.

The scent broadcasting means, graphic display means, sound broadcasting means and computer means (data reception, multimedia data storage) are integrated inside the multi-sensory picture. These various means are implemented by distinct physical modules, thus making them easy to manage (low-cost replacement, malfunction detection, etc.) The scent broadcasting means (3) can possibly be placed at a distance from the multi-sensory picture if it is desirable to broadcast the scents at a distance. It can also consist of a number of satellite scent diffusers of the multi-sensory picture, remotely controlled by the computer means (5) over a wired or wireless link.

In reference to FIGS. 4 and 5, the airflow (FA) produced by the electric turbine (10) passes through the containers of the scent broadcasting means (3). The air, enriched with scent molecules (odorous flow, OF) is directed outside the picture by ducts (11) then expelled through orifices (9). In the configuration shown in FIG. 4, the turbine delivers a vertical flow of air downwards so as to benefit from gravity to optimise power consumption in order to supply an airflow, the airflow passing through the means (3). The flow, which is always directed towards the bottom of the picture, is contained within the duct (1) then emitted under the picture towards the user by means of a duct (11) elbow (12).

For ease of production, this elbow (12) can be made at a right angle to the main duct (11).

In order to amplify the spectator's impression that the screen is scented, the airflow is expelled through the elbow at an angle α greater than zero upwards in the direction of the spectator. The angle α is chosen between 0 and 45°, for example 20°. However this value is determined, or even adapted, according to the proximity of the spectator (simple geometric calculations can provide an approximate value of the desired angle).

The elbow (12) is equipped with a reflective plate (13) which prevents the air flow from “crashing” against the lower part of the duct elbow. The elbow can also be rounded in order to solve this problem, in which case the plate (13) is not necessary.

The data-reception means (4) consist of a CD-ROM (Compact Disc—Read Only Memory) reader, a DVD (Digital Video Disc) reader, a USB (Universal Serial Bus) reader, or any other kind of data medium which can store the amount of data required for the multi-sensory works. It can also be a receiver for data downloaded from a remote server or from a wireless transmitter. In this case, the data are stored on a hard disk which is an integral part of the computer means (5). The download can be carried out over a temporary wired connection via a standard computer communications port, or over a wireless link using a known technique, for example a technique implementing the Wi-Fi or Bluetooth standard.

The computer means (5) consist mainly of a microprocessor, RAM memory, potentially a hard disk, and software. They control the screen (1), the sound broadcasting means (2), the scent broadcasting means (3), and potentially store the multi-sensory work (4) data. The interchangeable frame (6) has the same appearance as the moulded decorative frames commonly used to frame paintings. It is preferably made out of wood, although other materials may also be used, for example aluminium or PVC for multi-sensory works requiring the picture to have a modern appearance. In order to stimulate the user's sense of touch, the frame can contain raised patterns intended to be touched by the user. These shapes are designed in harmony with the images, sounds and scents, the whole making up the multi-sensory work.

According to a specific embodiment of the invention, the multi-sensory picture is equipped with a mechanism, for example a system with a controllable electric motor and gears, for automatically orienting the picture in so-called portrait or landscape formats, or even in an intermediary position which could be termed diagonal. The orientation is dynamically controlled by the computer means (5), according to commands contained in the multi-sensory work of art.

According to another embodiment of the invention, the software integrated with the computer means (5) is capable of counting the number of times each multi-sensory work is broadcast, making it possible to calculate the royalties owed to the creators of the works.

The digital work can be presented in the form of an MPEG (Moving Picture Experts Group) compressed video flow complete with an index for controlling the scent broadcasting means or any other type of means connected to the device (picture orientation in relation to the broadcast images, etc.) Data is recorded at every video scene change (represented by an “I” (Intra) image) to indicate the scents to be broadcast, as well as possibly parameters regulating this broadcast (time, power, synchronisation, timing of the mix of several scents, etc.). The computer means (5) are capable of decoding these complementary data in order to effectively control all the broadcasting means.

According to another embodiment of the invention, the program integrated in the computer means (5) is capable of decrypting the data of the multi-sensory work if they have been previously encrypted. On the one hand, this prevents the work from being partially broadcast on a standard multimedia viewing device and, on the other hand, it authorises or forbids the broadcast of a multi-sensory work by simply managing the distribution of decryption keys.

According to another embodiment of the invention, the multi-sensory picture is equipped with means of communicating with a large network, such as the internet, which makes it possible to download multi-sensory works from a remote server.

According to another embodiment of the invention, several multi-sensory pictures, synchronised with each other by a wired or a wireless link, are placed together in a single room. This makes it possible to broadcast multi-sensory works of art designed for a multiplicity of multi-sensory pictures, for example works wherein the images are displayed sequentially on one of the pictures and then on another, which contributes to increasing the attention of the spectator.

In reference to FIG. 5, the screen (1) is mounted on a door (14) by means of hinges and hydraulic power cylinders, the supply means (power and data) of the screen consisting, for example, of an electric braid located at the joint. Opening the door stops broadcasting the multi-sensory work (stops the video, sound, scents, etc.) by detection using suitable sensors. This opening grants access to the internal elements of the device, namely the computer means (5) for programming, updating, etc. and the scent broadcasting means (3) for changing the containers. The user can therefore easily and intuitively access the scent containers in order to replace them if necessary.

Microcontroller or computer means detect that the door is open and act on the scent broadcasting means (3) in order to
place the all the mechanical elements of the scent broadcasting means in a neutral position so that the user cannot make a false move and alter them. For example, servomotors are placed in the position in which the containers are hermetically sealed.
lock access to the scent containers/cartridges with a view to ensuring the safety of chemical substances. For example, the user cannot simply remove the containers from their sites.

1- Multi-sensory picture characterised in that it comprises:
graphic display means (1),
sound broadcasting means (2),
means of broadcasting a plurality of scents (3),
digital data-reception means (4),
computer means (5),
said computer means being arranged so as to control and synchronise said display (1), sound broadcasting (2) and scent broadcasting (3) means.

2. Multi-sensory picture according to claim 1, characterised in that it also comprises a frame that can be removed and exchanged, allowing it to be personalised according to the digital multi-sensory work broadcast.

3- Multi-sensory picture according to claim 1 or 2, characterised in that the scent broadcasting means work by a method of micronising an odorous liquid, liquids with different scents being placed in different refillable or replaceable vials.

4- Multi-sensory picture according to claim 1 or 2, characterised in that the scent broadcasting means work by causing an airflow to pass through a series of porous balls previously soaked in odorous liquids, balls with different scents being placed in different refillable or replaceable containers.

5- Multi-sensory picture according to any one of the claims from 1 to 4, characterised in that the sound source of the sound broadcasting means is mostly concealed and emits the sound in the direction of a rigid plate forming a reflector (8), allowing the sound to propagate towards the spectator without having to pass through the picture.

6- Multi-sensory picture according to any one of the claims from 1 to 5, characterised in that it comprises means of downloading the digital multi-sensory work to be broadcast, by using a wired or wireless link of known type.

7- Multi-sensory picture according to any one of the claims from 1 to 6, characterised in that the computer means comprise a program for counting the number of times the multi-sensory works are broadcast, with a view to calculating the royalties owed to their creators.

8- Multi-sensory picture according to any one of the claims from 1 to 7, characterised in that it comprises mechanical means that automatically orient the picture in portrait, landscape or diagonal format, controlled by the computer means according to commands contained in the digital multi-sensory work.

9- Multi-sensory picture according to any one of the claims from 1 to 8, characterised in that the computer means comprise software for decrypting previously encrypted digital multi-sensory works, so as to prevent a multi-sensory work from being broadcast to users not owning the software decryption key.

10- Multi-sensory picture according to any one of the preceding claims, characterised in that is comprises means (11) for carrying a flow of air loaded with scent molecules through said scent broadcasting means (3), said duct means (11) comprising an outlet orifice (9) located below said screen (1) and pointing substantially upwards.

11- Multi-sensory picture according to the preceding claim, characterised in that said outlet orifice (9) is at an angle greater than zero in relation to the horizontal, upwards in the direction of a spectator.

12- Multi-sensory picture according to any one of the preceding claims, characterised in that said screen (1) is mounted on a door (14) mounted such as to pivot in relation to the frame (6), which comprises all the computer (5), scent broadcasting (3), sound broadcasting (2) and digital data-reception means.

13- Multi-sensory picture according to the preceding claim, characterised in that it comprises means capable of placing said scent broadcasting means (3) in neutral position and locking said containers of said scent broadcasting means (3) when the door (14) is open in relation to said frame (6).

14- System made up of a plurality of multi-sensory pictures according to any one of the claims from 1 to 13, communicating over a standard telecommunications network with a remote computer server, making it possible to broadcast in different digital multi-sensory works in several locations from a single server.

15- Device made up of a plurality of multi-sensory pictures according to any of the claims from 1 to 13, communicating with each other by means of a wired or wireless link, making it possible to broadcast multi-picture multi-sensory works in the same room.

16- Software database intended to design multi-sensory works intended for multi-sensory picture according to any one of the claims from 1 to 15.
